(19) United States
(12) Patent Application Publication
(10) Pub. No.: US 2021/0196746 A1
(43) Pub. Date: Jul. 1, 2021

(54) TREATMENT OF COVID-19

(71) Applicant: Judith Boston, Manhattan Beach, CA (US)

(72) Inventor: Judith Boston, Manhattan Beach, CA (US)

(21) Appl. No.: 16/911,290

(22) Filed: Jun. 24, 2020

Related U.S. Application Data
(63) Continuation-in-part of application No. 16/727,764, filed on Dec. 26, 2019.
(60) Provisional application No. 63/032,269, filed on May 29, 2020.
(60) Provisional application No. 63/030,115, filed on May 26, 2020.
(60) Provisional application No. 63/024,412, filed on May 13, 2020.
(60) Provisional application No. 63/023,986, filed on May 13, 2020.
(60) Provisional application No. 63/009,990, filed on Apr. 14, 2020.
(60) Provisional application No. 62/984,732, filed on Mar. 3, 2020.
(60) Provisional application No. 62/975,562, filed on Feb. 12, 2020.

(51) Int. Cl. A61K 33/00; A61P 31/14; A61K 9/51

(52) U.S. Cl. CPC A61K 33/00; A61K 9/0085; A61K 9/5153; A61P 31/14

(57) ABSTRACT

This disclosure relates to the use of an oxygen-containing liquid for treating a coronavirus infection, such as Covid-19.

### CROSS-REFERENCE TO RELATED APPLICATIONS

This application is a continuation-in-part of application Ser. No. 16/727,764, filed Dec. 26, 2019. This application also claims the benefit of Provisional Application Ser. No. 63/032,269, filed May 29, 2020; Provisional Application Ser. No. 63/030,115, filed May 26, 2020; Provisional Application Ser. No. 63/024,412, filed May 13, 2020; Provisional Application Ser. No. 63/023,986, filed May 13, 2020; Provisional Application Ser. No. 63/009,990, filed Apr. 14, 2020; Provisional Application Ser. No. 63/002,917, filed Mar. 31, 2020; Provisional Application Ser. No. 62/984,732, filed Mar. 3, 2020; and Provisional Application Ser. No. 62/975,562, filed Feb. 12, 2020. All of these priority documents are incorporated by reference herein in their entirety.

### BACKGROUND

SARS-CoV-2 is a viral infection resulting in the disease CoVID 19. In some countries the virus has completely overtaken the medical system. The manifestations of this infection range from asymptomatic to severe. In its severe form CoVID 19 results in acute respiratory distress that may progress to death from hypoxemic respiratory failure. Though the number may vary depending on geographic location and other factors, it is estimated that up to 5% of the diagnosed cases end up in intensive care units. Complicating this number is the use of various oxygen delivery systems. Depending on the type and fit of mask, some of the virus particles may be aerosolized. This may result in infection of medical staff which may further complicate the overwhelmed medical systems.

### SUMMARY

This disclosure relates to the use of an oxygen-containing liquid for treating conditions related to ischemia, hypoxia, an alteration in electrochemistry, VEGF, HIF, or a reactive oxygen species, cancer, and other conditions.

Some embodiments include a method of treating an ocular condition comprising administering or delivering an oxygen-containing liquid to the eye of a mammal suffering from an ocular condition.

### BRIEF DESCRIPTION OF FIGURES

FIG. 1 depicts the scotopic b-wave response of ischemic rabbit eyes subjected to treatment with a hyperbaric oxygen solution as compared to controls.

FIG. 2 depicts the levels of VEGF in retinal pigment epithelium (RPE) cells exposed to hypoxic conditions and treated with a hyperbaric oxygen solution.

FIG. 3 depicts the levels of HIF in RPE cells exposed to hypoxic conditions and treated with a hyperbaric oxygen solution.

FIG. 4 is a schematic diagram of the ACE 2 pathway.

FIG. 5 depicts two possible embodiments of nanoparticles containing an oxygen-containing liquid.

### DETAILED DESCRIPTION

This disclosure relates to methods of treating ischemic conditions, such as ocular ischemic conditions, other conditions related to hypoxia, or conditions related to reactive oxygen species, comprising administering or delivering an oxygen-containing liquid to a mammal, such as a human being, for the treatment of the condition.

The term “treating” or “treatment” broadly includes any kind of treatment activity, including the diagnosis, cure, mitigation, or prevention of disease in man or other animals, or any activity that otherwise affects the structure or any function of the body of man or other animals.

The oxygen-containing liquid may be any liquid composition containing oxygen, or a compound that provides an oxygen pressure to a liquid, or a liquid which provides oxygen to mammalian cells or tissue, which is suitable for use in a mammal, including a human being, for therapeutic purposes. The oxygen-containing liquid may be aqueous, or may be based upon a suitable organic solvent, or may be a combination of aqueous and organic solvents. The liquid may be in the form of a solution, or a multiple phase liquid, such as a suspension, a colloid, an emulsion, a shear-thinning gel, etc. For many routes of administration, such as injections, it may be important for the oxygen-containing liquid to be sterile.

An oxygen-containing liquid may be formulated for any desirable route of delivery including, but not limited to, parenteral, suppository, intravenous, intradermal (e.g. intradermal injection), subcutaneous, oral, inhalative, metered dose inhaler (MDI), transdermal, topical to an eye (e.g. eye drops for delivery to the anterior segment of the eye or eyedrops for delivery to the posterior segment of the eye) or to skin, transmucosal, rectal, intravaginal, intraperitoneal, intramuscular, intralesional, intranasal, subcutaneous (e.g. subcutaneous injection), buccal, intraocular injection, intravitreal injection, sub-retinal injection, intrathecal injection (e.g. directly into the heart), etc. The term “injection” includes injection of a pharmaceutical composition, insertion of an implant or drug delivery device, as well as other types of injections.

In some embodiments, rather than being directly administered, an oxygen-containing liquid may be generated in the target tissue by inserting an implant or drug delivery device into or near the target tissue, which could provide long term delivery of the oxygen-containing liquid. For example, the implant could comprise a biodegradable or bioerodible polymer having components of an oxygenating composition dispersed in the polymer. As the polymer degrades or erodes, the components of the oxygenating composition will mix in the aqueous environment of the tissue into which the implant is inserted, thus generating an oxygen-containing liquid at or near the tissue to be targeted. The implant or device may be administered by any route described above, including intravenously (e.g. by injection), intravitreally (e.g. by injection), or subretinally (e.g. by injection). Oxygen-containing liquid may also be generated by other types of solid devices, such as punctal plugs and contact lenses containing components of the oxygenating composition, which gradually diffuse out of the devices. Alternatively, a punctal plug or contact lens might be biodegradable or bioerodible.

Any therapeutic composition, dosage form, drug delivery device, implant, etc. described herein (e.g. by oral, IV, etc.) may be formulated or designed for timed release, delayed release, controlled release, sustained release, etc., e.g. by the use of biodegradable polymers or bioerodible materials. A composition, dosage form, drug delivery device, implant, etc., may provide targeted delivery by both the route and/or location of administration and by including materials or structural features that are designed to respond to the specific environment, chemical properties, chemical activity, biological properties, or biological activity of a type of cell, tissue, organ, or biological system to be targeted

Examples of materials with timed release, delayed release, controlled release, sustained release, etc. properties include silica-based materials, or organic biodegradable materials, such as polymers comprising poly (D,L-lactic acid) (PLA) and poly (D,L-lactic-co-glycolic acid) or poly (lactic-co-glycolic acid) (PLGA), polyesteramide (PEA, DSM chemical), and polycaprolactone (PCL); hydrogels, such as polyvinyl alcohols (PVA), PEG amines, PEG-N-hydroxysuccinamide esters and the like; collagen based materials; acrylic acid and methacrylic acid copolymers and various esters thereof, e.g. methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polyacrylamide, poly(methacrylic acid anhydride), and glycidyl methacrylate copolymers; polymerizable quaternary ammonium compounds, e.g. quaternized aminoalkyl esters and aminoalkyl amides of acrylic acid and methacrylic acid, for example β-methacryloxyethyltrimethylammonium methosulfate, β-acryloxypropyltrimethylammonium chloride, and trimethylaminomethylmethacrylamide methosulfate, etc. The quaternary ammonium atom can also be part of a heterocycle, as in methacryloxyethylmethylmorpholinium chloride or the corresponding piperidinium salt, or it can be joined to an acrylic acid group or a methacrylic acid group by way of a group containing hetero atoms, such as a polyglycol ether group. Further suitable polymerizable quaternary ammonium compounds include quaternized vinyl-substituted nitrogen heterocycles such as methyl-vinyl pyridinium salts, vinyl esters of quaternized amino carboxylic acids, styryltrialkyl ammonium salts, and the like. Other polymerizable quaternary ammonium compounds include benzyldimethylammoniumethylmethacrylate chloride, diethylmethylammoniumethyl-acrylate and -methacrylate methosulfate, N-trimethylammoniumpropylmethacrylamide chloride, and N-trimethylammonium-2,2-dimethylpropyl-1-methacrylate chloride.

In some embodiments, a delivery system, such as a nanoparticle delivery system (e.g. PLGA nanoparticles), may provide extended delivery of the oxygen containing liquid to a patient. For example, the delivery system may provide oxygen to the target cells, tissue, organ, or system (e.g. IV nanoparticles, such as PLGA nanoparticles, containing the oxygen containing liquid, may provide oxygen to the blood) over an extended period of time, such as at least about 1 hour, at least about 4 hours, at least about 8 hours, at least about 12 hours, at least about 24 hours, at least about 2 days, at least about 3 days, at least about 4 days, at least about 5 days, at least about 6 days, at least about 7 days, at least 2 weeks, at least 3 weeks, at least 4 weeks, at least 6 weeks, at least 2 months, at least 3 months, at least 4 months, at least 6 months, about 1-2 days, about 2-4 days, about 4-7 days, about 1-2 weeks, about 2-4 weeks, about 1-2 months, about 2-4 months, about 4-6 months, or about 1-4 months. In some embodiments, administration of the delivery system may be repeated at an interval of about 4-8 hours, about 8-16 hours, about 16-24 hours, about 1-2 days, about 2-4 days, about 4-7 days, about 1-2 weeks, about 2-4 weeks, about 1-2 months, about 2-4 months, about 4-6 months, or about 1-4 months.

In some embodiments, the delivery system is sterile.

In some embodiments, an oxygen containing liquid, or a device or implant that releases or generates the oxygen containing liquid in the body, is directly administered, e.g. by injection, insertion or other means, into a tissue, an organ or body system affected by a condition to be treated, such as an organ or system infected by SARS-CoV-2 or affected by COVID-19, such as the central nervous system such as, the brain, eyes, nerves; the cardiopulmonary system, such as the heart, lungs, etc.; the liver; the kidneys; musculoskeletal system, the muscles; the endocrine system, such as the pancreas; the gastrointestinal system, such as the esophagus, the stomach, the small intestine, the large intestine, etc.

The oxygen-containing liquid may have a higher partial oxygen pressure than plain water, for example, at room temperature (e.g. 23° C.) or body temperature (e.g. 37° C.), the oxygen-containing liquid may have an oxygen pressure that is at least 120 mmHg, at least 140 mmHg, at least 145 mmHg, at least 150 mmHg, at least 155 mmHg, at least 160 mmHg, at least 165 mmHg, at least 170 mmHg, up to 180 mmHg, up to 200 mmHg, up to about 250 mmHg, up to about 300 mmHg, up to about 350 mmHg, up to about 400 mmHg, up to about 450 mmHg, up to about 500 mmHg, about 120-500 mmHg, about 20-40 mmHg, about 40-60 mmHg, about 60-80 mmHg, about 80-100 mmHg, about 100-120 mmHg, about 120-140 mmHg, about 140-145 mmHg, about 145-150 mmHg, about 150-155 mmHg, about 155-160 mmHg, about 160-165 mmHg, about 165-170 mmHg, about 170-175 mmHg, about 175-180 mmHg, about 140-150 mmHg, about 150-160 mmHg, about 160-170 mmHg, about 170-180 mmHg, about 180-190 mmHg, about 190-200 mmHg, about 200-210 mmHg, about 210-220 mmHg, about 220-230 mmHg, about 230-240 mmHg, about 240-250 mmHg, about 250-260 mmHg, about 260-270 mmHg, about 270-280 mmHg, about 280-290 mmHg, about 290-300 mmHg, about 300-320 mmHg, about 320-340 mmHg, about 340-360 mmHg, about 360-380 mmHg, about 380-400 mmHg, about 400-420 mmHg, about 420-440 mmHg, about 440-460 mmHg, about 460-480 mmHg, about 480-500 mmHg, about 140-160 mmHg, about 160-180 mmHg, about 180-200 mmHg, about 160-200 mmHg, about 200-250 mmHg, about 250-300 mmHg, about 300-350 mmHg, about 350-400 mmHg, about 400-450 mmHg, about 450-500 mmHg, about 140-200 mmHg, about 200-300 mmHg, about 300-400 mmHg, about 400-500 mmHg, 500-750 mmHg, 750-1,000 mmHg, 1,000-1,250 mmHg, 1,250-1,500 mmHg, about 175 mmHg, or any oxygen pressure in a range bounded by any of these values. In some embodiments, the oxygen-containing liquid is a hyperbaric oxygen solution (e.g. Examples 1-3 below).

While there may be many ways to add oxygen to a liquid, some oxygen-containing liquids may contain an oxygenating composition, such as a compound, or a combination of compounds, that release an oxygen gas, e.g. by a chemical reaction or chemical degradation. Suitable oxygenating compositions may contain metal oxides (such as CaO, MgO, etc.), metal hydroxides (such as Ca(OH)₂, Mg(OH)₂), peroxides (such as hydrogen peroxide or an organic peroxide), or combinations thereof. Other ingredients may be added to increase or reduce the rate of oxygen release, depending upon the particular need. For example, faster oxygen release may provide higher oxygen pressure. On the other hand, slower oxygen release may provide a longer, more consistent, or more sustained, oxygen pressure. Examples of suitable oxygenating compositions are described in U.S. Pat. No. 8,802,049, which is incorporated by reference herein in its entirety. One useful oxygenating composition contains about 20-30% Ca(OH)₂, about 10-15% H₂O₂, about 0.5-5% sodium acetate, about 0.5-5% KH₂PO₄, and about 1-20% Carrageenan, based upon the total weight of the oxygen-containing liquid. In some embodiments, the total amount of oxygen atoms present in all metal oxides, metal hydroxides, and peroxides present in the oxygen-containing liquid is about 20-70%, about 20-50%, about 50-70%, about 20-30%, about 30-40%, about 40-50%, about 50-60%, about 60-70%, about 70-90%, or about 80-95% of the total weight of the oxygen-containing liquid.

As mentioned above, the components of these oxygenating compositions, such as metal oxides, metal hydroxides, and/or peroxides, may be dispersed in a bioerodible or biodegradable polymer, such as a silicon-based polymer, a polyester, a polyorthoester, a polyphosphoester, a polycarbonate, a polyanhydride, a polyphosphazene, a polyoxalate, a poly(amino acid), a polyhydroxyalkanoate, a polyethyleneglycol, a polyvinylacetate, a polyhydroxyacid, a polyanhydride, or copolymer or blend thereof (e.g. a co-polymer of lactic and glycolic acid).

Appropriate excipients for use in an oxygen-containing liquid may include, for example, one or more carriers, binders, fillers, vehicles, tonicity agents, buffers, disintegrants, surfactants, dispersion or suspension aids, thickening or emulsifying agents, preservatives, lubricants and the like or combinations thereof, as suited to a particular dosage from desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. This document is incorporated herein by reference in its entirety.

In addition to solvent, oxygen, and/or oxygenating compositions, a liquid dosage form for IV, injection (e.g. intraocular injection, sub-retinal injection, intrathecal, directly into the heart), topical (e.g. to an eye), or oral administration to a mammal, including a human being, may contain excipients such as bulking agents (such as mannitol, lactose, sucrose, trehalose, sorbitol, glucose, raffinose, glycine, histidine, polyvinylpyrrolidone, etc.), tonicity agents (e.g. dextrose, glycerin, mannitol, sodium chloride, etc.), buffers (e.g. acetate, e.g. sodium acetate, acetic acid, ammonium acetate, ammonium sulfate, ammonium hydroxide, citrate, tartrate, phosphate, triethanolamine, arginine, aspartate, benzenesulfonic acid, benzoate, bicarbonate, borate, carbonate, succinate, sulfate, tartrate, tromethamine, diethanolamine etc.), preservatives (e.g. phenol, m-cresol, a paraben, such as methylparaben, propylparaben, butylparaben, myristyl gamma-picolinium chloride, benzalkonium chloride, benzethonium chloride, benzyl alcohol, 2-phenoxyethanol, chlorobutanol, thimerosal, phenylmercuric salts, etc.), surfactants (e.g. polyoxyethylene sorbitan monooleate or Tween 80, sorbitan monooleate polyoxyethylene sorbitan monolaurate or Tween 20, lecithin, a polyoxyethylene-polyoxypropylene copolymer, etc.), solvents (e.g. propylene glycol, glycerin, ethanol, polyethylene glycol, sorbitol, dimethylacetamide, Cremophor EL, benzyl benzoate, castor oil, cottonseed oil, N-methyl-2-pyrrolidone, PEG, PEG 300, PEG 400, PEG 600, PEG 600, PEG 3350, PEG 400, poppyseed oil, propylene glycol, safflower oil, vegetable oil, etc.) chelating agents (such as calcium disodium EDTA, disodium EDTA, sodium EDTA, calcium versetamide Na, calteridol, DTPA), or other excipients.

A liquid dosage form comprising an oxygen-containing liquid, e.g. for IV, injection (e.g. intraocular injection, sub-retinal injection, etc.), topical (e.g. to an eye), or oral administration, to a mammal, including a human being, may have any suitable pH, such as about 2-12, about 2-4, about 4-6, about 6-8, about 8-10, about 10-12, about 6-7, about 7-8, about 8-9, about 6-6.5, about 6.5-7, about 7-7.5, about 7.5-8, about 8-8.5, about 8.5-9, about 7-7.2, about 7.2-7.4, about 7.4-7.6, about 7.6-7.8, about 7.8-8, or any pH in a range bounded by any of these values.

For many routes of administration, it may be helpful for the oxygen-containing liquid to be hypertonic or hyperosmolar, e.g. having a tonicity or an osmolarity greater than about 290 mOsm/L, such as about 290-600 mOsm/L, about 290-400 mOsm/L, about 400-500 mOsm/L, or about 500-600 mOsm/L; isotonic or isoosmolar, e.g. having a tonicity or an osmolarity near that of the body tissue to which it administered, such as about 290 mOsm/L, about 250-350 mOsm/L, about 250-320 mOsm/L, about 270-310 mOsm/L, or about 280-300 mOsm/L; or hypotonic or hypoosmolar, e.g. having tonicity or an osmolarity less than about 290 mOsm/L, such as about 150-290 mOsm/L, about 150-200 mOsm/L, about 200-290 mOsm/L, about 200-250 mOsm/L, or about 250-290 mOsm/L.

An oxygen-containing liquid may also potentially be delivered in nanoparticle delivery systems, nanoemulsion delivery systems, microemulsions delivery systems, microsomal delivery systems, liposomal delivery systems, or lysosomal delivery systems. For example, an oxygen containing liquid might be contained in a reverse micelle or inside a nanoparticle, nanoemulsion, microemulsion, microsome, liposome, or lysosome. In some embodiments, the oxygen containing liquid is contained within polymer nanoparticles (such as PLGA nanoparticles).

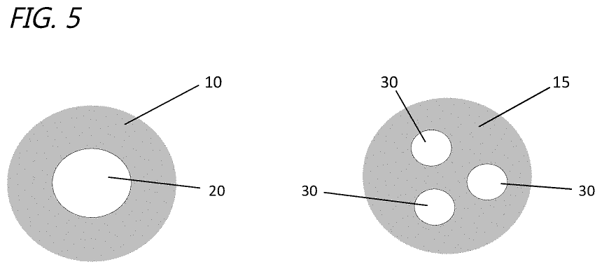

For example, as shown in FIG. 5, polymer 10 (e.g. PLGA) may form an exterior solid layer around oxygen-containing liquid center 20, or several smaller liquid nanodroplets or nanoparticles 30 may be encased in a larger solid polymer (e.g PLGA) host nanoparticle 15.

In addition to the above, it may be desirable for an orally administered liquid to contain a sweetening agent such as sucrose or saccharin; or a flavoring agent such as peppermint, methyl salicylate, or orange flavoring.

For creams, gels, ointments, etc. it may be desirable to include thickening agents, such as polyethylene glycol, polyacrylic acid, cetyl alcohol, stearyl alcohol, carnauba wax, stearic acid, hydroxyethylcellulose, guar gum, locust bean gum, xanthan gum, gelatin, silica, bentonite, magnesium aluminum stearate, etc.

A liquid dosage form comprising an oxygen-containing liquid might be part of a pharmaceutical product, which comprises the oxygen-containing liquid, an oxygen sensor, and a drug dispensing device. In some embodiments, the oxygen-containing liquid can only be dispensed if the oxygen-containing liquid has the desired oxygen pressure, such as an oxygen pressure described above.

While any suitable oxygen sensor may be used, a high performance microsensor available from Unisense is an example of a useful oxygen sensor.

Any suitable drug dispensing device may be used, such as a syringe or other form of injection device, a drop dispensing device.

Hypoxia, ischemia and reactive metabolites contributes to development and exacerbation of many disease states. The common denominator resulting in inhibition of tissue repair is tissue hypoxia.

Facilitating delivery of oxygen to tissues can result in adjunct and direct treatments in a wide variety of medical conditions.

Tissue hypoxia is low tissue oxygen level, usually related to impaired circulation. Tissue hypoxia, ischemia and reactive metabolites contribute to development and exacerbation of many disease states.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the Hypoxic Induction Factor (HIF) level of the tissue (e.g. eye tissue) having ischemia to be decreased by at least about 10%, at least about 20%, at least about 30%, at least about 40%, at least about 50%, at least about 60%, at least about 70%, at least about 80%, at least about 90%, or more as compared to the HIF level of the tissue (e.g. eye tissue) having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the HIF level of the tissue (e.g. eye tissue) having ischemia to be decreased so that it is within about 50%, within about 40%, within about 30%, within about 20%, within about 10%, within about 5%, within about 3%, or within about 1% of the HIF level of non-ischemic tissue (e.g. the contralateral eye).

In some embodiments, the reduction of the HIF level of the tissue may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, the reduction of the HIF level of the tissue may be continue for at least 1 day, at least 2 days, at least 3 days, at least 4 days, at least 5 days, at least 6 days, at least 7 days, at least 14 days, at least 21 days, or at least 28 days.

Administering or delivering an oxygen-containing liquid, such as a hyperbaric oxygen-containing liquid, to a mammal may be used to treat any type of ischemic condition, such as wounds, vasculopathies, malignant tumors, arthritis, atherosclerotic plaques, cancers, tumors, burns, inflammatory conditions, including inflammation of neural tissue (e.g. concussion).

In some embodiments, the ischemic condition is an ocular condition, such as diabetic retinopathy, macular degeneration, macular edema, diabetic macular edema, glaucoma, sickle eye disease, ocular inflammation, hypertensive retinopathy, ocular ischemic syndrome, branched retinal vein occlusion, branched retinal artery occlusion, central retinal vein occlusion, central retinal artery occlusion, retinal detachment, penetrating globe injury, traumatic optic neuropathy, optic neuritis, an inflammatory ocular condition, etc. In some embodiments, the ocular ischemic condition is diabetic retinopathy.

In some embodiments, the ocular ischemic condition is macular degeneration. In some embodiments, the ocular ischemic condition is diabetic macular edema. In some embodiments, the ocular ischemic condition is glaucoma. In some embodiments, the ocular ischemic condition is sickle cell eye disease. In some embodiments, the ocular ischemic condition is an ocular inflammation. In some embodiments, the condition is hypertensive retinopathy. In some embodiments, the condition is ocular ischemic syndrome. In some embodiments, the condition is retinal vein occlusion. In some embodiments, the condition is arterial occlusion, e.g. in the retina. In some embodiments, the condition is branched retinal vein occlusion. In some embodiments, the condition is branched retinal artery occlusion. In some embodiments, the condition is central retinal vein occlusion. In some embodiments, the condition is central retinal artery occlusion. In some embodiments, the condition is retinal detachment. In some embodiments, the condition is penetrating globe injury. In some embodiments, the condition is traumatic optic neuropathy. In some embodiments, the condition is optic neuritis. In some embodiments, the condition is an inflammatory ocular condition.

In some embodiments, the ischemic condition is one wherein the electrochemistry is altered, such as heart attack, stroke, neural ischemia, injury to the central nervous system, traumatic brain injury, spinal injury, acute and chronic traumatic encephalopathy, and immunocytotoxicity. Administering or delivering an oxygen-containing liquid, such as a hyperbaric oxygen-containing liquid, may also be useful to treat diseases or conditions related to, or caused by, sun damage or oxidation.

In some embodiments, an oxygen containing liquid may be used for the treatment of cancer. For example, the oxygen containing liquid may be administered in conjunction with a chemotherapy agent such as an alkylating agent, an antimetabolite, an anti-tumor antibiotic, a topoisomerase inhibitor, a mitotic inhibitor, etc. In some embodiments, co-administration of an oxygen containing with a chemotherapy drug can help to improve the activity of the chemotherapy drug or radiation therapy. For example, an oxygen containing liquid may increase oxygen within cancer and tumors to improve the effectiveness of radiation therapy and/or chemotherapy. An oxygen containing liquid may be used to reduce or halt tumor growth and/or growth rate. In some embodiments, a chemotherapy drug may be administered in an aqueous solution, e.g. intravenously or injected into the site of the cancer. An oxygen containing liquid may also have other therapeutic effects for the treatment of cancer.

Other conditions that may be treated with an oxygen containing liquid include anemia, migraine headaches, refectory osteomyelitis, a coronavirus infection (such as SARS-CoV-2, which causes COVID-19), a viral infection, a bacterial infection, etc.

Oxygenation of a hypoxemic Coronavirus/CoVID 19 patient may be improved by administering an oxygen containing liquid to the patient. It is believed that an intravenous oxygen containing solution can help the body overcome hypoxemia and ameliorate organ damage. Intravenous oxygen delivery is a unique method of oxygenation. Intravenous oxygenation will allow faster recovery and help heal organs, avoiding the sometimes devastating consequences of hypoxemia from CoVID 19.

As disclosed herein, it is been discovered that an oxygen containing liquid can ameliorate consequences of retinal hypoxia. In vitro and in vivo studies have been conducted with some measure of success. It has been found that some oxygen containing liquids are safe across multiple cell lines. As described herein, oxygen containing liquids may protect cells in vitro and modulate the Vascular Endothelial Growth Factor (VEGF) response of cells exposed to a hypoxic environment (see Example 2 below). Additionally, as illustrated in Example 1 below, it has been demonstrated that some oxygen containing liquids can facilitate recovery of cellular function after induced ischemia.

Viral growth may be inhibited by high oxygen levels. Gene expression may be altered by oxidative conditions in SARS-CoV-2. Hypoxia may directly or indirectly modulate viral response. SARS-CoV-2, like other viruses, may target the hypoxic pathway and glycolytic metabolism. These mechanisms are complex and involve the Hypoxic Induction Factor (HIF) pathway and Vascular Endothelial Growth Factor (VEGF) regulation and how these relate to DNA or RNA viruses. It thus appears that perhaps the characteristics of CoVID19 may be understood in the context of oxygen level in the body.

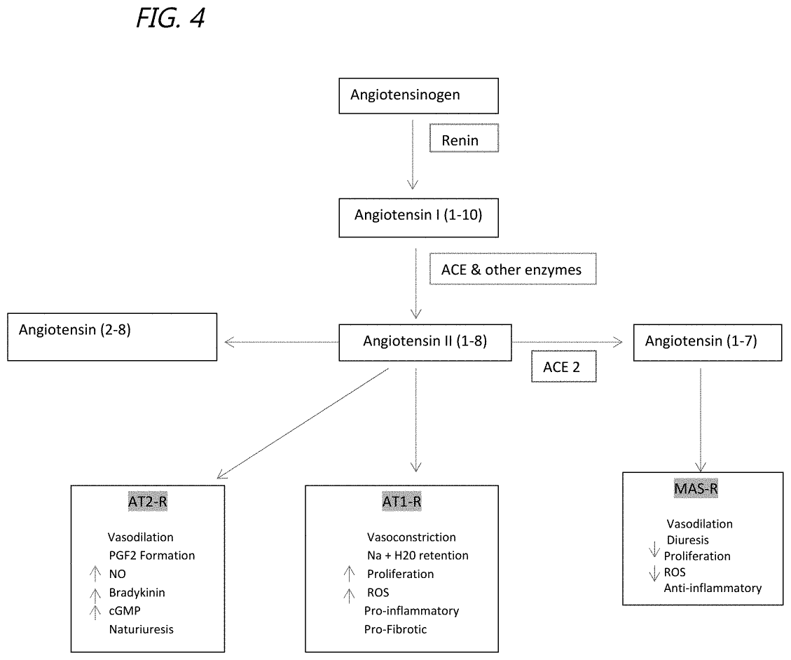

It has been determined that CoVID 19 binds the ACE2 pathway which activates the MAS receptor via Angiotensinogen. (See FIG. 4.) It is believed that a paucity of ACE2 receptor availability may result from SARS-CoV-2 binding of ACE2 receptors. Less availability of ACE2 receptors may result in loss of the protective aspects from MAS receptor activation. This limitation may result in a “loop” in which increased reactive oxygen species and increased inflammation can adversely affect the body thereby worsening the effects of the viral infection.

Some patients present with sepsis. A characteristic of sepsis is lactic acidosis. We can potentially measure this lactic acid and viral load and further characterize CoVID 19. With an intravenous oxygen liquid lactic acidosis may potentially be overcome.

SARS-CoV-2/COVID 19 may be characterized using parameters measured by blood gas machines and other devices to measure oxygen (O₂), oxygen partial pressure (PO₂), carbon dioxide partial pressure (PCO₂), lactic acid, pH, electrolytes (such as Na⁺, K⁺, Ca²⁺, Mg²⁺, HCO₃⁻, CO₃²⁻, H₂PO³⁻, HPO₃²⁻, PO₃³⁻, etc.), and others. Blood gas machines and other devices may also be used to determine hypoxia and reactive oxygen species (ROS) in blood, such as superoxide, hydrogen peroxide, hydroxy radical, etc. Measured indexes of hypoxia, ROS and electrolyte variances in the blood can be used to characterize the disease severity, prognosis and staging. These blood tests can be used as markers in an algorithm which may affect treatment decisions.

Based upon the indices above, CoVID 19 may potentially be treated and/or severity of the disease may potentially be ameliorated, and/or the progress of the disease may potentially be halted by intravenously administering an oxygen containing liquid. Reduction of organ damage and correction of hypoxemia and lactic acidosis may lessen the severity of the disease and/or halt virus propagation. This will afford the body a better opportunity to heal itself. This is especially true in conditions in which the level of hypoxia can indicate the severity of the disease.

Determining viral load may potentially be used as an indication of severity of infection. Based on viral load and indices of blood gas, blood measurements and hypoxia, CoVID 19 may potentially be categorized into subcategories/subunits which will then provide guidance for treatment.

A coronavirus infection, such as COVID-19 caused by SARS-CoV-2, may be treated by administering an oxygen-containing liquid directly, e.g. by direct administration of the liquid or a form containing a liquid, such as a gel, cream, or a liquid phase dispersed in a solid (e.g. a biodegradable polymer). Treatment may also occur by indirect administration of an oxygen-containing liquid, such as by directly administering a solid or other non-liquid material that forms an oxygen containing liquid in situ, such as in a tissue or organ to be treated, in a gastrointestinal tract, or in another bodily fluid. In some embodiments, an oxygen-containing liquid is administered by intravenous injection, or by intravenous injection of a solid or other non-liquid material that forms an oxygen containing liquid in the patient's body after intravenous injection.

A coronavirus infection, such as COVID-19, may be treated by directly or indirectly administering an oxygen-containing liquid, to reduce the level of reactive oxygen species, reduce the level of free radicals, reduce an inflammatory response, reduce generalized inflammation, reduce cytokine storm, reduce hypoxia, protect against hypoxia, reduce or normalize VEGF levels, reduce ischemia, reduce ischemic damage, improve recovery from ischemic damage, ameliorate organ damage, help to heal organs, improve ACE2 receptor activity, interfere with virus binding to receptors modified by hypoxia, interfere with virus binding (e.g. by the spike protein) to ACE2 receptors modified by hypoxia, correct metabolic abnormalities, correct hematologic oxygen deficits and/or hematologic abnormalities, facilitate repair and restoration of disease impacted organs due to hypoxia or other metabolic or disease induced organ damage, correct neurologic oxygen deficits, correct respiratory oxygen deficits, increase MAS receptor activation, reduce lactic acidosis, reduce sepsis, reduce the virus in the patient, eliminate the virus, reduce viral propagation, slow viral propagation, reduce virus pathogenicity, etc., in a COVID-19 patient to improve the patient's health. In some embodiments, directly or indirectly administering an oxygen-containing liquid to treat a coronavirus infection, such as COVID-19, may be useful to normalize VEGF in the lungs, which may help to reduce or prevent scarring in the patient.

For treatment of viral infections, such as coronavirus infection, such as SARS-CoV-2/COVID-19, an oxygen containing liquid may be used alone, or in combination with a vaccine. For some vaccines, oxygenation may help to activate the vaccine or improve the efficacy of the vaccine.

In summary, in vivo and in vitro studies of oxygen containing liquids have shown that oxygen containing liquids can normalize increased VEGF levels in hypoxic environments, can be protective of cells exposed to a hypoxic environment, have excellent safety in vitro using 3 cell lines, and facilitate recovery from ischemic damage. Because of this, it is believed that administering an oxygen containing liquid can potentially reduce virus propagation and/or pathogenicity. It is also believed that administering an oxygen containing liquid can potentially supplement the human body's basic requirement of oxygen facilitating the body's own natural mechanisms to heal. Blood gas measurements, potentially with use of an algorithm, might be used to determine severity of disease before oxygen is required. Mobile blood gas machines are available.

An oxygen-containing liquid may also be administered to a mammal who is undergoing gene therapy, and may improve the outcome of the gene therapy. An oxygen-containing liquid may also be administered to a mammal in conjunction with treatment with stem cells, such as stem cells in the eye, e.g. retina, optic nerve, or other ocular structures.

An oxygen-containing liquid may also be administered to a mammal for improvement in blood oxygenation. This may be measured by transcutaneous oxygen measurement, pulse oximetry, or blood gas measurement.

An oxygen-containing liquid may also be administered to a mammal for improvement in vitreoretinal oxygenation, oxygenation of retina, oxygenation of subretina, or a combination thereof.

Improvement in many of the conditions described herein may be measured by optical coherent tomography (OCT), optical coherent tomography angiography, angiography, retinal oximetry, or some other imaging technique. Administering or delivering an oxygen-containing liquid, such as a hyperbaric oxygen-containing liquid, to a mammal may also be used to improve blood oxygen level in chronic diseases and to reduce the need for blood transfusions.

Administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, may result in an increase in ERG function of the ischemic tissue. For example, the scotopic b-wave response of an eye having ischemia may be about 0-5 mV, about 5-10 mV, about 10-15 mV, about 15-20 mV, about 20-50 mV, about 50-100 mV, or about 100-120 mV.

In some embodiments, administering or delivering an oxygen-containing liquid, such as a hyperbaric oxygen-containing liquid, to a mammal suffering from an ocular ischemic condition results in the scotopic b-wave response of the eye having ischemia to be increased by at least about 20 mV, at least about 30 mV, at least about 40 mV, at least about 50 mV, at least about 60 mV, at least about 70 mV, at least about 80 mV, at least about 90 mV, at least about 100 mV, or more, as compared to the scotopic b-wave response of the eye having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the scotopic b-wave response of the tissue (e.g. eye tissue) having ischemia to be increased by at least 2-fold, 3-fold, 4-fold, 5-fold, 6-fold, 7-fold, 8-fold, 9-fold, 10-fold, or more as compared to the scotopic b-wave response of the tissue (e.g. eye tissue) having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the scotopic b-wave response of the tissue (e.g. eye tissue) having ischemia to be increased so that it is within about 50%, within about 40%, within about 30%, within about 20%, within about 10%, within about 5%, within about 3%, or within about 1% of the scotopic b-wave response of normal or non-ischemic tissue (e.g. the contralateral eye).

In some embodiments, the improvement in ERG function may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the visual acuity of the mammal (e.g. human being) to be increased by about 10%, about 20%, about 30%, about 50%, about 70%, about 90%, or so that it is within about 50%, within about 40%, within about 30%, within about 20%, within about 10%, within about 5%, within about 3%, or within about 1% of the visual acuity of a normal eye (e.g. the contralateral eye).

In some embodiments, the improvement in visual acuity may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the retinal thickness of the mammal (e.g. human being) to be decreased by about 10%, about 20%, about 30%, about 50%, about 70%, about 90%, or so that it is within about 50%, within about 40%, within about 30%, within about 20%, within about 10%, within about 5%, within about 3%, or within about 1% of the retinal thickness of a normal eye (e.g. the contralateral eye).

In some embodiments, the improvement in retinal thickness may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in VEGF, HIF, electrochemistry, or a reactive oxygen species, such as an ocular condition related to ischemia, hypoxia, an alteration in VEGF, HIF, electrochemistry, or a reactive oxygen species, results in the neovascularization of the mammal (e.g. human being) to be reduced by about 10%, about 20%, about 30%, about 50%, about 70%, about 90%, or so that it is within about 50%, within about 40%, within about 30%, within about 20%, within about 10%, within about 5%, within about 3%, or within about 1% of the neovascularization of a normal eye (e.g. the contralateral eye).

In some embodiments, the improvement in neovascularization may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the Vascular Endothelial Growth Factor (VEGF) level of the tissue (e.g. eye tissue) having ischemia to be decreased by at least about 10%, at least about 20%, at least about 30%, at least about 40%, at least about 50%, at least about 60%, at least about 70%, at least about 80%, at least about 90%, or more as compared to the VEGF level of the tissue (e.g. eye tissue) having ischemia immediately prior to administration of the oxygen-containing liquid.

In some embodiments, administering or delivering an oxygen-containing liquid, such as a hyperbaric oxygen-containing liquid, to a mammal suffering from a condition related to ischemia, hypoxia, an alteration in electrochemistry, VEGF, HIF, or a reactive oxygen species, such as an ocular condition related to ischemia, hypoxia, an alteration in electrochemistry, VEGF, HIF, or a reactive oxygen species, results in the VEGF level of the tissue (e.g. eye tissue) having ischemia to be decreased so that it is within about 50%, within about 40%, within about 30%, within about 20%, within about 10%, within about 5%, within about 3%, or within about 1% of the VEGF level of normal or non-ischemic tissue (e.g. the contralateral eye).

In some embodiments, the reduction in the VEGF level of the tissue may be observed within 1 day, within 2 days, within 3 days, within 4 days, within 5 days, within 6 days, within 7 days, within 14 days, within 21 days, within 28 days, within 2 months, within 3 months, within 4 months, within 6 months, within 1 year, or longer.

The following embodiments are specifically contemplated.

Embodiment D-1. A method of detecting infection by a corona virus, or detecting the progress of the disease, comprising: obtaining a blood level of a chemical species in a human being in need thereof.

Embodiment D-2. The method of Embodiment D-1, wherein the chemical species is a gas.

Embodiment D-3. The method of Embodiment D-2, wherein the gas is O₂.

Embodiment D-4. The method of Embodiment D-2 or D-3 wherein the gas is CO₂.

Embodiment D-5. The method of Embodiment D-1, D-2, D-3, or D-4, wherein the chemical species is lactic acid.

Embodiment D-6. The method of Embodiment D-1, D-2, D-3, D-4, or D-5, wherein the chemical species is H⁺, OH⁻, H₃O⁺, or a combination thereof, as determined by blood pH.

Embodiment D-7. The method of Embodiment D-1, D-2, D-3, D-4, D-5, or D-6, wherein the chemical species is an electrolyte.

Embodiment D-8. The method of Embodiment D-7, wherein the electrolyte is Nat Embodiment D-9. The method of Embodiment D-7 or D-8, wherein the electrolyte is K⁺.

Embodiment D-10. The method of Embodiment D-7, D-8, or D-9, wherein the electrolyte is Ca²⁺.

Embodiment D-11. The method of Embodiment D-7, D-8, D-9, or D-10, wherein the electrolyte is Mg²⁺.

Embodiment D-12. The method of Embodiment D-7, D-8, D-9, D-10, or D-11, wherein the electrolyte is HCO₃⁻.

Embodiment D-13. The method of Embodiment D-7, D-8, D-9, D-10, D-11, or D-12, wherein the electrolyte is CO₃²⁻.

Embodiment D-14. The method of Embodiment D-7, D-8, D-9, D-10, D-11, D-12, or D-13, wherein the electrolyte is a phosphate species.

Embodiment D-15. The method of Embodiment D-7, D-8, D-9, D-10, D-11, D-12, D-13, or D-14, wherein the chemical species is a reactive oxygen species.

Embodiment D-16. The method of Embodiment D-15, wherein the reactive oxygen species comprises superoxide.

Embodiment D-17. The method of Embodiment D-15 or D-16, wherein the reactive oxygen species comprises hydrogen peroxide.

Embodiment D-18. The method of Embodiment D-15, D-16, or D-17, wherein the reactive oxygen species comprises hydroxy radical.

Embodiment D-19. Treatment of a corona virus by administering a therapy to a patient with a level of the chemical species of Embodiment D-1, D-2, D-3, D-4, D-5, D-6, D-7, D-8, D-9, D-10, D-11, D-12, D-13, D-14, D-15, D-16, D-17, or D-18 above a threshold value.

Embodiment D-20. Treatment of a corona virus by administering a therapy to a patient with a level of the chemical species of Embodiment D-1, D-2, D-3, D-4, D-5, D-6, D-7, D-8, D-9, D-10, D-11, D-12, D-13, D-14, D-15, D-16, D-17, or D-18 above a threshold value.

Embodiment D-21. The method of Embodiment D-19 or D-20, wherein the treatment comprises administering an oxygen-containing liquid to the patient.

Embodiment D-22. The method or treatment of Embodiment D-1, D-2, D-3, D-4, D-5, D-6, D-7, D-8, D-9, or D-10, further comprising determining a viral load in the human being.

Embodiment D-23. A method of detecting infection by a corona virus, or detecting the progress of the disease, comprising: determining a viral load in a human being in need thereof.

Embodiment 1. A method of treating a mammal suffering from a condition related to ischemia, hypoxia, an alteration in electrochemistry, VEGF, HIF, or a reactive oxygen species, comprising delivering an oxygen-containing liquid to the mammal suffering from the condition, wherein the treatment results in a therapeutic effect on the condition.

Embodiment 2. The method of embodiment 1, wherein the condition is ocular and the oxygen-containing liquid is delivered to the eye of the mammal.

Embodiment 3. The method of embodiment 1 or 2, wherein the oxygen-containing liquid has an oxygen pressure that is higher than 140 mmHg.

Embodiment 4. The method of embodiment 1, 2, or 3, wherein the oxygen-containing liquid contains a compound that releases an oxygen gas.

Embodiment 5. The method of embodiment 1, 2, 3, or 4, wherein the oxygen-containing liquid has an osmolarity of about 250 mOsm/L to about 350 mOsm/L.

Embodiment 6. The method of embodiment 1, 2, 3, 4, or 5, wherein the oxygen-containing liquid comprises a metal oxide.

Embodiment 7. The method of embodiment 1, 2, 3, 4, 5, or 6, wherein the oxygen-containing liquid comprises a metal hydroxide.

Embodiment 8. The method of embodiment 1, 2, 3, 4, 5, 6, or 7, wherein the oxygen-containing liquid comprises a peroxide.

Embodiment 9. The method of embodiment 1, 2, 3, 4, 5, 6, or 8, wherein the oxygen-containing liquid is sterile.

Embodiment 10. The method of embodiment 1, 2, 3, 4, 5, 6, 7, 8, or 9, wherein the treatment results in an improvement of ERG function within 1 week of administering the oxygen-containing liquid to the eye of the mammal.

Embodiment 11. The method of embodiment 1, 2, 3, 4, 5, 6, 7, 8, 9, or 10, wherein the treatment results in a reduction in VEGF expression within 1 week of administering the oxygen-containing liquid to the eye of the mammal.

Embodiment 12. The method of embodiment 1, 2, 3, 4, 5, 6, 7, 8, 9, 10, or 11, wherein the condition is diabetic retinopathy.

Embodiment 13. The method of embodiment 1, 2, 3, 4, 5, 6, 7, 8, 9, 10, or 11, wherein the condition is macular degeneration.

Embodiment 14. The method of embodiment 1, 2, 3, 4, 5, 6, 7, 8, 9, 10, or 11, wherein the condition is diabetic macular edema.

Embodiment 15. The method of embodiment 1, 2, 3, 4, 5, 6, 7, 8, 9, 10, or 11, wherein the condition is sickle cell eye disease.

Embodiment 16. The method of embodiment 1, 2, 3, 4, 5, 6, 7, 8, 9, 10, or 11, wherein the condition is an ocular inflammation.

Embodiment 17. The method of embodiment 1, 2, 3, 4, 5, 6, 7, 8, 9, 10, or 11, wherein the condition is hypertensive retinopathy.

Embodiment 18. The method of embodiment 1, 2, 3, 4, 5, 6, 7, 8, 9, 10, or 11, wherein the condition is ocular ischemic syndrome.

Embodiment 19. The method of embodiment 1, 2, 3, 4, 5, 6, 7, 8, 9, 10, or 11, wherein the condition is branched retinal vein occlusion.

Embodiment 20. The method of embodiment 1, 2, 3, 4, 5, 6, 7, 8, 9, 10, or 11, wherein the condition is branched retinal artery occlusion.

Embodiment 21. The method of embodiment 1, 2, 3, 4, 5, 6, 7, 8, 9, 10, or 11, wherein the condition is central retinal vein occlusion.

Embodiment 22. The method of embodiment 1, 2, 3, 4, 5, 6, 7, 8, 9, 10, or 11, wherein the condition is central retinal artery occlusion.

Embodiment 23. The method of embodiment 1, 2, 3, 4, 5, 6, 7, 8, 9, 10, or 11, wherein the condition is retinal detachment.

Embodiment 24. The method of embodiment 1, 2, 3, 4, 5, 6, 7, 8, 9, 10, or 11, wherein the condition is penetrating globe injury.

Embodiment 25. The method of embodiment 1, 2, 3, 4, 5, 6, 7, 8, 9, 10, or 11, wherein the condition is traumatic optic neuropathy.

Embodiment 26. The method of embodiment 1, 2, 3, 4, 5, 6, 7, 8, 9, 10, or 11, wherein the condition is optic neuritis.

Embodiment 27. The method of embodiment 1, 2, 3, 4, 5, 6, 7, 8, 9, 10, or 11, wherein the condition is an inflammatory ocular condition.

Embodiment 28. The method of embodiment 1, 2, 3, 4, 5, 6, 7, 8, 9, 10, 11, 12, 13, 14, 15, 16, 17, 18, 19, 20, 21, 22, 23, 24, 25, 26, or 27, wherein the oxygen-containing liquid is injected into an eye of a human being.

Embodiment 29. The method of embodiment 1, 2, 3, 4, 5, 6, 7, 8, 9, 10, 11, 12, 13, 14, 15, 16, 17, 18, 19, 20, 21, 22, 23, 24, 25, 26, or 27, wherein the oxygen-containing liquid is topically administered to a human being.

Embodiment 30. The method of embodiment 1, 2, 3, 4, 5, 6, 7, 8, 9, 10, 11, 12, 13, 14, 15, 16, 17, 18, 19, 20, 21, 22, 23, 24, 25, 26, or 27, wherein the oxygen-containing liquid is orally administered to a human being.

### Example 1

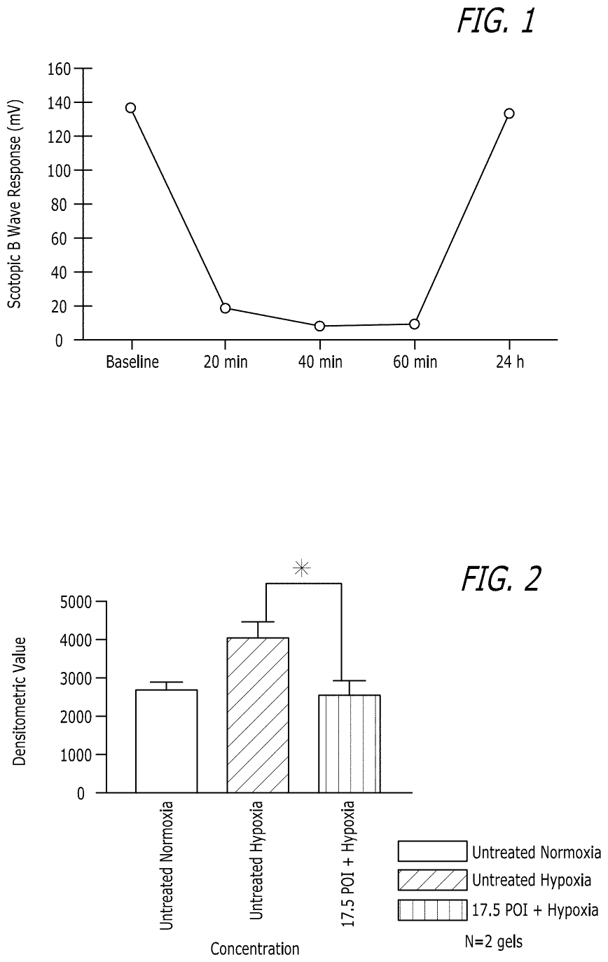

The effect of a hyperbaric oxygen solution in ischemic rabbit eyes was evaluated. Ischemia was induced in six rabbits as follows. A needle was connected to a saline bag, which was elevated to create pressure at the needle opening. The needle was placed into the rabbit eye and the intraocular pressure was allowed rise in the rabbit eyes for 90 minutes, which caused ischemia in the rabbit eyes. Rabbit 1 initially received no treatment, but then received an intraocular injection of the hyperbaric oxygen solution an hour after the needle attached to the saline bag was removed. Rabbits 2-3 were intraocularly injected with normal saline (with an oxygen pressure of 112.6 mmHg) 20 minutes after the needle attached to the saline bag was removed. Rabbits 4-6 were intraocularly injected with a hyperbaric oxygen solution (with an oxygen pressure of 175.2 mmHg) 20 minutes after the needle attached to the saline bag was removed. The results are depicted in Table 1 and FIG. 1.

TABLE 1

| | | Scotopic B Wave Response (mV) | | | | |
|---|---|---|---|---|---|---|
| | | | 20 min | 40 min | 60 min | 24 hr |
| | Treat- | Base- | after | after | after | after |
| Rabbit | ment | line | ischemia | ischemia | ischemia | ischemia |
| 1 | None | 136.9 | 18.52 | 7.8 | 8.85 | 133.4 |
| 2 | Saline | 155.4 | 16.65 | 12.03 | — | |
| 3 | Saline | 192.8 | 33.76 | 15.6 | 12.32 | |
| 4 | Hyper- | 136.1 | 11.46 | 22.17 | 25.8 | |
| | baric | | | | | |
| | oxygen | | | | | |
| 5 | Hyper- | — | 19.43 | 28.4 | 48.6 | |
| | baric | | | | | |
| | Oxygen | | | | | |
| 6 | Hyper- | — | 50.4 | 75.07 | 94.45 | |
| | baric | | | | | |
| | Oxygen | | | | | |

### Example 2

ARPE-19 cells were treated with a hyperbaric oxygen solution (oxygen pressure of 175.2 mmHg) and placed into a hypoxic chamber for 48 hours. Control cells were incubated in the hypoxic chamber without the hyperbaric oxygen solution. Phase contrast images show that the hypoxic ARPE-19 cells rounded up and showed unusual morphology compared to the hyperbaric oxygen treated hypoxic cells. There were 71 rounded cells per high power field in the control hypoxic cells versus 8 rounded cells per high power field in the hyperbaric oxygen solution treated hypoxic cells. It was concluded that hyperbaric oxygen solution appears to protect cells from the typical damage that results from exposure to hypoxia.

### Example 3

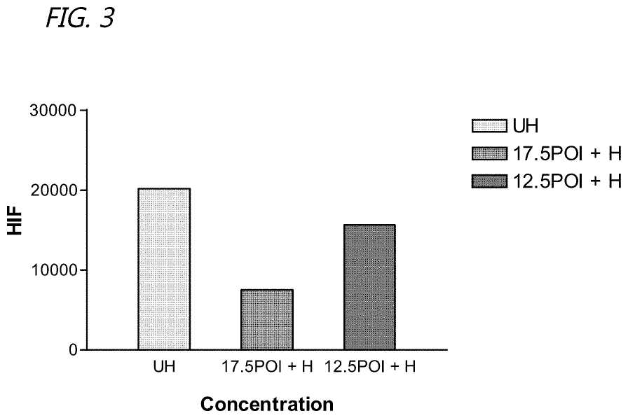

Retinal pigment epithelium cells were exposed to hypoxic conditions for 48 hours. Treatment with a hyperbaric oxygen solution (oxygen pressure of 175.2 mmHg) resulted in a statistically significant reduction in cellular levels of expressed vascular endothelial growth factor (VEGF) p<0.05 (FIG. 2) and HIF (FIG. 3).

As shown in FIG. 2, with 17.5% of an oxygenating ingredient added, the VEGF level of cells that had been exposed to hypoxic conditions (17.5POI+Hypoxia) was lower than the HIF level of cells that had been exposed to hypoxic conditions without treatment (Untreated Hypoxia), and was comparable to cells that had not been exposed to hypoxic conditions (Untreated Normoxia).

HIF level was analyzed by Western blot analyses. Proteins were extracted from the cell cultures and the protein concentrations measured with BCA protein Assay Reagent Kit (Pierce, Rockford, Ill.) according to the manufacturer's protocol.

As shown in FIG. 3, with 12.5% of an oxygenating ingredient added, the HIF level of cells that had been exposed to hypoxic conditions (12.5POI+H) was lower than the HIF level of cells that had been exposed to hypoxic conditions without treatment (UH). Furthermore, with 17.5% of an oxygenating ingredient added, the HIF level of cells that had been exposed to hypoxic conditions (17.5POI+H) was even lower.

These results indicate that treatment with a hyperbaric oxygen solution normalizes the VEGF and HIF levels of cells exposed to hypoxic conditions so that they are similar to the basal level of VEGF and HIF.

1. A method of interfering with binding of a SARS-CoV-2-Spike protein to an ACE2 receptor or reducing propagation of a SARS-CoV-2 virus, comprising administering an oxygen containing liquid to a human being, wherein the oxygen containing liquid contains an oxygenating composition that releases oxygen gas by a chemical reaction or chemical degradation.

2. The method of claim 1, wherein the oxygen containing liquid is administered intravenously.

3. (canceled)

4. The method of claim 1, wherein the oxygen containing liquid is directly administered to the central nervous system of the human being.

5. The method of claim 1, wherein the oxygen containing liquid is directly administered to the brain of the human being.

6. The method of claim 1, wherein the oxygen containing liquid is directly administered to an eye of the human being.

7. The method of claim 1, wherein the oxygen containing liquid is directly administered to the cardiopulmonary system of the human being.

8. The method of claim 1, wherein the oxygen containing liquid is directly administered to the heart of the human being.

9. The method of claim 1, wherein the oxygen containing liquid is directly administered to a lung of the human being.

10. The method of claim 1, wherein the oxygen containing liquid is directly administered to the liver of the human being.

11. The method of claim 1, wherein the oxygen containing liquid is directly administered to a kidney of the human being.

12. The method of claim 1, wherein the oxygen containing liquid is directly administered to the musculoskeletal system of the human being.

13. The method of claim 1, wherein the oxygen containing liquid is directly administered to a muscle of the human being.

14. The method of claim 1, wherein the oxygen containing liquid is directly administered to the endocrine system of the human being.

15. The method of claim 1, wherein the oxygen containing liquid is directly administered to the pancreas of the human being.

16. The method of claim 1, wherein the oxygen containing liquid is directly administered to the gastrointestinal system of the human being.

17. A method of treating Covid-19, comprising administering an oxygen containing liquid to a human being in need thereof, wherein the oxygen containing liquid is contained within poly (lactic-co-glycolic acid) nanoparticles or microparticles, wherein the oxygen containing liquid contains an oxygenating composition that releases oxygen gas by a chemical reaction or chemical degradation.

18. The method of claim 17, wherein the oxygen containing liquid contained within poly (lactic-co-glycolic acid) nanoparticles is administered intravenously.

19. A method of treating Covid-19 comprising administering an oxygen containing liquid to a human being in need thereof, wherein the oxygen containing liquid is prepared by dissolving or dispersing an oxygenating composition in an aqueous liquid, wherein the oxygenating composition releases oxygen gas by a chemical reaction or chemical degradation.

20. The method of claim 19, wherein the oxygen containing liquid is administered intravenously.

21. The method of claim 1, wherein the oxygen containing liquid has a temperature that is between room temperature and body temperature when it is administered to the human being.
